(19) United States
(12) Patent Application Publication
(10) Pub. No.: US 2004/0158144 A1
(43) Pub. Date: Aug. 12, 2004

(54) NMR probe particularly useful for intra-luminal imaging

(75) Inventors: Hanan Keren, Kfar Sava (IL); Avram B. Simon, London (GB)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: TopShooter Medical IMRI Inc.

(21) Appl. No.: 10/769,772

(22) Filed: Feb. 3, 2004

Related U.S. Application Data
(60) Provisional application No. 60/444,188, filed on Feb. 3, 2003.

(51) Int. Cl. A61B 5/05

(52) U.S. Cl. 600/423; 128/899

(57) ABSTRACT

An NMR probe, includes a permanent magnet generating a static magnetic gradient field (B₀), and a pair of RF coils enclosing the permanent magnet for receiving RF current to generate a varying magnetic field (B₁) perpendicular to the static magnetic gradient field (B₀). The pair of RF coils are orthogonally located with respect to each other such that the gradient magnetic field (B₁) is rotatable about the longitudinal axis of the probe according to the current through one RF coil relative to that through the other RF coil. As described, the NMR probe is particularly useful for intra-luminal imaging.

### FIELD AND BACKGROUND OF THE INVENTION

The present invention relates to an NMR (Nuclear Magnetic Resonance) probe. The invention is particularly useful in constructing such probes for intra-luminal imaging, and is therefore described below with respect to such applications.

Examinations aimed at detecting stenosis and blockage in coronary arteries or other blood vessels are usually made by detecting obstructions to blood flow, for example, by injecting a contrast material into the blood vessel and detecting such material by X-ray angiography. However, the information obtained in such procedures is incomplete because neither the nature nor the composition of the obstructing plaque, nor its azimuthal distribution, is determined.

Therefore, there is an urgent need for a means to supplement such diagnostic procedures by measuring the azimuthal distribution of plaque in a narrowed blood vessel, and by determining whether the plaque contains a dangerously large amount of lipids.

Magnetic Resonance Imaging (MRI) is increasingly being used for producing high resolution images of the interiors of human bodies. However, conventional MRI apparatus generally includes a very large magnet producing a very strong, homogenous magnetic field, and radio frequency (RF) gradient coils for producing, in each of the three main axes, weaker, linear gradient fields in which the field strength varies in a linear manner along a particular axis. The patient is placed in the homogenous main field; and when the linear gradient field by the RF coils is added, nuclei at different positions will process at different resonance frequencies. The resonance frequency of the signal from a nucleus is proportional to the field strength, which in turn is proportional to the distance between the point of interest and the gradient coil. Therefore, the position of the nucleus can be determined by imposing gradients in different directions and detecting the frequencies of the signals. The conventional MRI apparatus, however, is large and bulky, and therefore is generally used only for imaging entire sections of humans, e.g., of the brain or a part of the torso.

Many types of NMR probes are described in the patent literature, such as in U.S. Pat. Nos. 5,296,811, 5,334,937, 5,390,673, 5,432,446, 5,572,132, 6,263,229, 6,377,048 and 6,516,213. However, insofar as known to the inventors, none of the known probes is capable of measuring the azimuthal distribution of plaque in a narrowed blood vessel, of for determining whether the plaque contains a dangerously large amount of lipids.

### OBJECTS AND BRIEF SUMMARY OF THE PRESENT INVENTION

An object of the present invention is to provide an NMR probe which can be implemented in a very small and compact construction so as to be useful in many of the applications for which the conventional MRI apparatus is not suitable. Another object of the invention is to provide an NMR probe particularly useful for intra-luminal imaging.

According to a broad aspect of the present invention, there is provided an NE probe, comprising: a permanent magnet generating a homogenous magnetic field (B₀); and a pair of RF gradient coils enclosing the permanent magnet for receiving RF current to generate a gradient magnetic field (B₁) perpendicular to the homogenous magnetic field (B₀); the pair of RF coils being orthogonally located with respect to each other such that the gradient magnetic field (B₁) is rotatable about the longitudinal axis of the probe according to the current through one RF coil relative to that through the other RF coil.

According to further features in the described preferred embodiments, the RF coils are located along the outer surface of the permanent magnet orthogonal to each other and have longitudinal axes parallel to the longitudinal axis of the probe.

According to still further features in the described preferred embodiments, the permanent magnet and the pair of RF coils are sized and configured for introduction into a lumen of a person's body. The basic principles involved which enable such a probe to be implemented in a small and compact construction for introduction into a lumen of a perfsons' body are more particularly described below in the section titled Basic Construction and Operation of the Novel Probe.

A number of embodiments of the invention are described below for purposes of example. In some described embodiments, the permanent magnet generates a diametrical magnetic field (B₀); in other described embodiments, it generates an axial-magnetic field; and in still other described embodiments, it generates a radial magnetic field.

In many of the described embodiments, the permanent magnet is of a permanent magnetic material for the complete length of the probe. However, other embodiments are described wherein the permanent magnet includes an end section of a permanent magnetic material at each of the opposite ends of the probe, and an intermediate section of an unsaturated, high-permeability material between the end sections. The latter construction enables a number of additional advantages to be attained, as will also be more particularly described below.

Further features and advantages of the invention will be apparent from the description below.

### BRIEF DESCRIPTION OF THE DRAWINGS

The invention is herein described, by way of example only, with reference to the accompanying drawings, wherein:

FIG. 1 illustrates one form of NMR probe constructed in accordance with the present invention;

FIG. 2 is a top plan view of the probe of FIG. 1 showing the generated magnetic fields;

FIG. 3 is a graph illustrating an example of how the magnetic field varies as a function of distance from the magnet surface;

FIG. 4 schematically illustrates the electrical circuit for energizing the two RF coils in the probe of FIG. 1;

FIG. 5 illustrates an NMR probe constructed in accordance with the present invention for use in intra-luminal imaging;

FIG. 6 illustrates the manner of using the probe of FIG. 5 in intra-luminal imaging;

FIGS. 7-11 illustrate a number of variations in the construction of the permanent magnet that may be used in the probe of FIGS. 1-6; and

FIGS. 12-17 illustrate examples of other variations in the construction of the permanent magnet to include a section of an unsaturated high-permeability material.

It is to be understood that the foregoing drawings, and the description below, are provided primarily for purposes of facilitating understanding the conceptual aspects of the invention and various possible embodiments thereof, including what is presently considered to be a preferred embodiment. In the interest of clarity and brevity, no attempt is made to provide more details than necessary to enable one skilled in the art, using routine skill and design, to understand and practice the described invention. It is to be further understood that the embodiments described are for purposes of example only, and that the invention is capable of being embodied in other forms and applications than described herein.

Basic Construction and Operation of the Novel Probe

The NMR probe in accordance with the present invention basically includes a permanent magnet generating a homogenous magnetic field (B₀), and a pair of RF gradient coils generating a gradient magnetic field (B₁) perpendicular to the homogenous magnetic field B₀, and orthogonally located with respect to each other such that the field B₁ is rotatable about the longitudinal axis of the probe according to the current through the two RF coils.

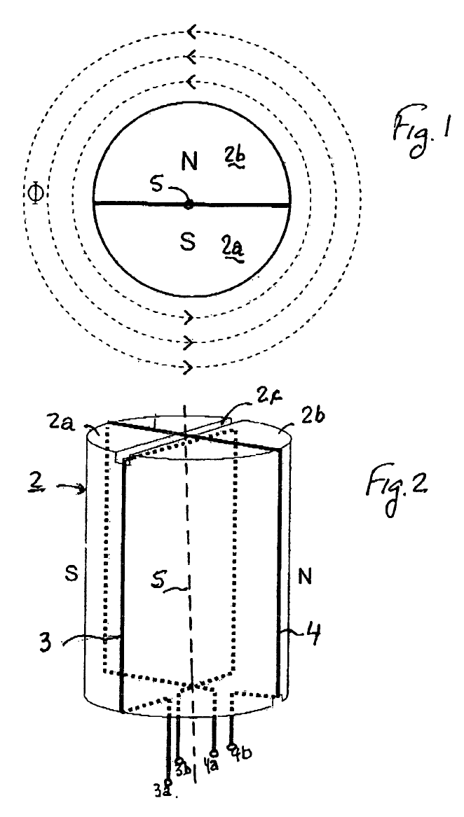

FIGS. 1 and 2 are perspective and plan views, respectively, illustrating an NMR probe constructed as described above. The illustrated probe includes a permanent magnet 2 of cylinder configuration, constituted of a half-cylinder 2a polarized as a South pole and another half-cylinder 2b polarized as a North pole. The illustrated probe further includes two RF coils 3, 4 enclosing the cylindrical permanent magnet 2 and orthogonally located with respect to each other, with the longitudinal axis of each coil concentric with the longitudinal axis 5 of the probe. As illustrated particularly in FIG. 1, the two RF coils 3, 4 enclose the cylindrical permanent magnet 2 and are received within slots, such as shown at 2c, in the permanent magnet.

The manner in which the NMP, probe illustrated in FIGS. 1 and 2 can be used f r imaging parts of the human body, particularly the interior of a lumen (such as a blood vessel), will be apparent from the following description:

The nuclei of the hydrogen atoms in the water and fat molecules within the human body (in particular those within the atherosclerotic plaque near the meaning device) are the source for the signal generated and measured by the NMR probe of the present invention. It is the energy between different quantum states of the nuclear spin which is the source of the NMR signal, and therefore the nuclei are sometimes denoted as ‘spins’ or ‘nuclear spins’. The creation and detection of NMR signals requires the presence of a magnetic field, and a coil (antenna), which is tuned to be able to transmit short and intense pulses at the resonance frequency of the spins and receive the signal emitted by the spins (at the same resonance frequency), following their excitation by the pulses.

There is a linear relation between the magnetic field and the resonance frequency:

ωₒ=−γBₒ [1]

Where ωₒ, is the resonance frequency (also called Larmor frequency), γ is the gyromagnetic ratio (characteristic of different isotopes in the periodic table), and Bₒ the magnetic field at the position of the nucleus. The vector notation in eq. [1] is derived from a classical description of the NMR phenomenon, whereby a nuclear spin has a magnetic moment that performs a precession around the axis defined by the direction of Bₒ. A large ensemble of spins is then described by a macroscopic magnetization vector, Mₒ, the direction of which (at equilibrium) is parallel to Bₒ. The direction of Bₒ is usually defined as the Z-axis and the scalar quantity Bₒ then represents the field component along the Z-axis.

A typical NMR measurement consists of applying short and intense pulses that (in the classical description) tilt the magnetization front its equilibrium position (along Z) towards the X-Y plane. In the absence of an external perturbation (i.e., after the pulse(s) is (are) turned off), the spin system will strive to return to thermal equilibrium. This means that the longitudinal component of the magnetization (M_{z}) will grow towards its equilibrium value (→Mₒ) at a characteristic rate 1/T₁, while the transverse components (Mₓ, M_{y}) will decay towards their equilibrium value (→0) at a characteristic rate 1/T₂. During this return to equilibrium, the magnetization vector will also perform a precession around the Z-axis (at the Larmor frequency) and this precession can induce a voltage in a suitably tuned and oriented coil. It is often the case (although this is not compulsory) that the same coil is used for both transmission of the excitation pulses and reception of the signal. In any case, when a current is passed through this coil, it will generate a magnetic field B₁ and it is necessary that the vector B₁ is perpendicular to Bₒ.

The essence of an NMR measurement is in the analysis and interpretation of the signal, generated as described above and detected by the receiver coil. The magnitude of the signal, its time dependencies (decay and recovery), and its Fourier components can yield information about the molecular environment of the nuclei. In the well known application of Magnetic Resonance Imaging (MRI), the Fourier analysis of the signal reveals the spatial distribution of the spins, resulting in the generation of images.

As indicated earlier, in the clinical-diagnostic NMR-based applications practiced to date the patients need to be positioned within the magnetic field generated by an external magnet. It is a unique feature of the NMR probe of the present invention that the magnetic field is generated by the device itself, within the patient's body, using a small permanent magnet In addition to this magnet, the device also contains a set of two perpendicular coils for excitation and detection, which generate a field B₁ perpendicular to the Bₒ field of the permanent magnet.

By varying the proportions of the power applied to each of the coils, one can rotate the direction of B₁, thereby confining the excitation to predefined azimuthal directions around the blood vessel. Signal excitation and detection along different directions will be performed sequentially, thereby enabling an azimuthal mapping of the blood vessel narrowing. At each angular orientation, the novel NMR probe provides a radial measurement of the lipid content in the plaque and the blood vessel wall. This radial discrimination is possible by virtue of a strong magnetic field gradient, which extends from the surface of the magnet into the blood vessel wall.

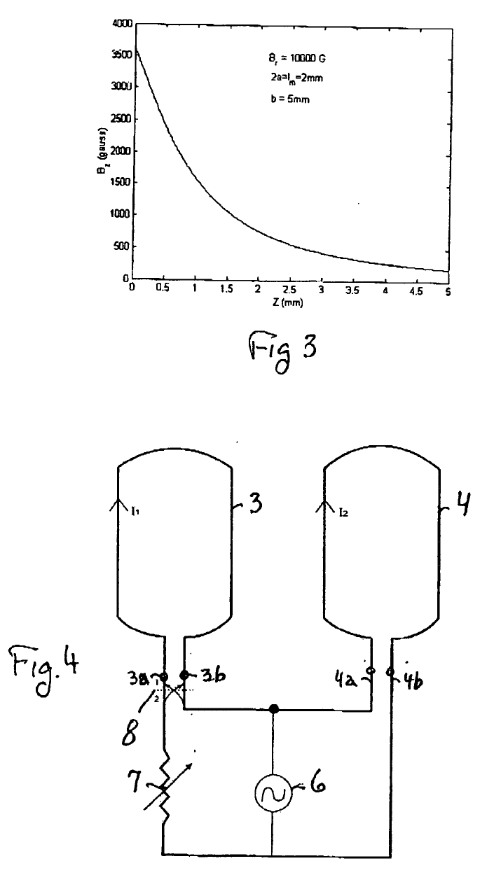

FIG. 3 shows an example of the magnetic field as a function of distance from the magnet surface, calculated for a permanent bar magnet, where Bᵣ is the residual magnetization, and a, b, lₘ are the magnet's width, height and length dimensions. As the graph of FIG. 3 shows, in close proximity to the magnet the field dependence is approximately linear with distance; thus the field dependence can be described in terms of a constant field gradient:

$$G = \frac{ⅆ {B}_{z}}{ⅆ z} .$$

The presence of such a gradient has two important consequences: First, due to the fact that the signal excitation with pulses of finite width is necessarily frequency-selective, the excitation will be confined to a narrow slice perpendicular to the direction of the gradient (i.e., perpendicular to Z). Secondly, the signal evolution (after excitation) will be strongly affected by translational motion of the nuclei (flow and diffusion). Both effects are described in more detail below:

Selective Volume Excitation:

The pulses applied to the spin system can be characterized by two parameters, namely their carrier frequency, (f_{c}=ω_{c}/2π); and their frequency bandwidth, Δf, which is determined by the pulse duration and its shape. The spins will be excited near a coordinate Z, at which the excitation frequency f_{c} corresponds to the Larmor frequency fₒ=ωₒ/2π. The Larmor frequency as function of Z can be defined by:

$${f}_{0} ( z ) = \frac{γ {B}_{z} ( z )}{2 π} = \frac{γ}{2 π} ( {B}_{z} ( 0 ) + {G}_{z} z ) \qquad [ 2 ]$$

Therefore, one can rearrange and find that the Z coordinate excited by a pulse with carrier frequency f_{c} will be given by:

$$z ( {f}_{c} ) = \frac{1}{{G}_{z}} ( \frac{2 π {f}_{0} ( z )}{γ} - {B}_{z} ( 0 ) ) \qquad [ 3 ]$$

The excited Z coordinates will be defined through the condition:

$$\begin{matrix}z ( {f}_{c} ) - \frac{Δ z}{2} \leq {z}_{exc} \leq z ( {f}_{c} ) + \frac{Δ z}{2} \\ where \text{:} & [ 4 ] \\ Δ z = \frac{2 πΔ f}{γ {G}_{z}} & [ 5 ]\end{matrix}$$

Attenuation of Spin Echo Signal by Diffusion in Field Gradient:

A spin echo is obtained following a (90°-τ-180°-) pulse sequence. The signal intensity at t=2τ, relative to the signal at t=0, is given by;

$$E ( 2 τ ) = exp ( \frac{2 τ}{{T}_{2}} - \frac{2}{3} D {γ}^{2} {G}^{2} {τ}^{3} ) \qquad [ 6 ]$$

where G is the field gradient and D is the molecular self diffusion coefficient. From the point of view of efficiency it is recommended to collect as many points a possible for each signal excitation. Therefore the signal will be collected and digitized not only at a single echo peak, as indicated in Eq. [6], but for a series of echoes which are created by the successive application of 180° pulses, according to the scheme: 90°-(τ-180°-τ)ₒ. In this case, the time dependence of the echo signal is:

$$E ( 2 n τ ) = exp ( \frac{2 n τ}{{T}_{2}} - \frac{2}{3} D {γ}^{2} {G}^{2} n {τ}^{3} ) \qquad [ 7 ]$$

From Eqs. [6] and [7] it is clear that the signal intensity is strongly dependent on the diffusion coefficient D. This dependence is exploited by the NMR probe of the present invention in order to collect signals whose intensity depends mainly on the presence of lipid-rich regions within the sensitive measurement region. There is evidence in the literature that the diffusion coefficient of the lipid molecules is significantly smaller than that of water molecules and, moreover, the diffusion coefficient of water molecules in lipid-rich regions is significantly smaller than that of water molecules in other environments.

It is therefore possible to conduct the procedure under conditions for which the signal from molecules, which are not in the lipid-rich athermanous core of a plaque, is attenuated so strongly that it will not generate a detectable signal. This will be achieved primarily through a judicious choice of the echo spacing parameter, τ. If, however, a measurable signal is detected under such conditions, it will unequivocally confirm the presence of a potentially dangerous lipid core. Moreover, the distribution of such a plaque around the vessel lumen will be determined from the directional sensitivity of the excitation, as described above.

General Scaling Considerations:

Evaluating the effective ‘slice widths’, ΔZ, that one could excite with a spectral bandwidth of 50 KHz (which is roughly the upper limit for excitation by short RF pulses), considering the spread in frequencies created by the field gradient. Assume that the S/N is proportional to the slice thickness. Actually, it should be proportional to a volume, i.e., we need to consider what happens along the other dimensions (there could be some higher order gradients), but let us ignore this for the sake of simplicity.

In this approximation, we can say that:

S∝a [8]

Where S is the sensitivity and a is the ‘typical’ length scale (in the above figures, a=1 mm). Therefore, we predict a linear loss in sensitivity when scaling down the dimension of a, but this could be compensated by the ‘filling factoe’ of the RF coils. If we take a circular single loop surface coil with radius a, in the X-Y plane, then the B₁ field perpendicular to the coil along the center is given by:

$${B}_{1} ( z ) ∝ I \frac{{a}^{2}}{{( {a}^{2} + {z}^{2} )}^{3 / 2}} \qquad [ 9 ]$$

Where is the current in the coil. Therefore, B₁(0)∝1/a, and since the sensitivity is proportional to B₁, this will exactly compensate the loss in sensitivity predicted by Eq. [1].

The Effect of Diffusion:

The signal attenuation of a spin echo in the presence of a constant gradient is given by:

$$E = exp ( \frac{2 τ}{{T}_{2}} - \frac{2}{3} D {γ}^{2} {G}^{2} {τ}^{3} ) \qquad [ 10 ]$$

where D is the diffusion coefficient, G is the gradient, and τ is the time between the 90 and 180 degree pulses (TE=2τ). The diffusion coefficient for water is about 0.002 mm²/s. The value of γG (for the 1 T magnet) is 6.7×10⁷ rps/mm. Let us assume that we require the attenuation factor inside the exponent not to exceed a value of 2. In this case we have the following limitations concerning the values of τ, G, and the field Bᵣ:

TABLE 1

| T(ms) | Max. G (G/cm) | Bᵣ (T) | B_{z} (z = 0) | ω₀ (MHz) |
|---|---|---|---|---|
| 0.1 | 14480 | 0.58 | 0.209 | 8.9 |
| 0.15 | 7882 | 0.315 | 0.114 | 4.8 |
| 0.2 | 5119 | 0.2 | 0.072 | 3.07 |
| 0.5 | 1295 | 0.052 | 0.0188 | 0.79 |
| 1 | 458 | 0.0183 | 0.0066 | 0.28 |

Therefore the choice of magnetic field will also depend, to a large extent, on the available RF power one can work with. The fact that one needs very short values of τ to overcome the large gradients will also dictate that the preferred mode of operation will be in the form of a Carr-Puroell-Meiboom-Gill echo train (90°ₓ-τ-180°_{y}-2x-180°_{y}- . . . ), where a data point is sampled at half the interval between successive 180° pulse pairs. The collection of data point along the echo train can be utilized for improving the S/N or to characterize the decay, in terms of diffusion and relaxation. The signal attenuation at the nth echo is given by:

$$E = exp ( \frac{2 n τ}{{T}_{2}} - \frac{2}{3} D {γ}^{2} {G}^{2} n {τ}^{3} ) \qquad [ 11 ]$$

The decay rate of this function can be made sensitive to the diffusion coefficient D, by changing τ while keeping nτ constant.

Scaling Down to Probe Size

We concluded that there should be no inherent loss in sensitivity, as the basic argument is that of the ‘filling factor’, i.e., the fact that the sensitivity of an RF (surface) coil increases inversely proportional to the coil's radius as can be seen in Eq. [2] of paragraph 3 above). This increase in sensitivity should compensate for the increase in the gradient (and concomitant decrease in slice thickness), which is expected when scaling down.

Taking into account that, for a small voxel, that change in size is along the 3 orthogonal dimensions X, Y and Z. We have to consider that a coil of radius a will excite and detect signal from a volume which is proportional to:

V∝a²(Δz) [12]

where (Δz) is the effective slice thickness, determined mainly by the local gradient, i.e., as we saw previously:

$$( Δ z ) ∝ \frac{1}{{G}_{z}} \qquad [ 13 ]$$

If we assume that the size of the magnet is of the same order as the size of the RF coil, then:

$${G}_{2} ∝ \frac{1}{a} \qquad [ 14 ]$$

Therefore by incorporating Eqs. [12-14] and combining it with Eq. [9], we arrive that the signal intensity (for Z→0) scales as:

$$S ∝ \frac{1}{a} • {a}^{2} • a = {a}^{2} \qquad [ 15 ]$$

where the first term comes from the B₁ dependence on the coil size, the second term from Eq. [8], and the third term from Eqs. [12-14].

Note that in this discussion we have not considered at all the effect of the gradient on diffusion attenuation (hopefully, we will be able to handle this by suitable shortening of the inter-echo separation τ). We have also ignored the fact that the B₁ field decreases as one moves away from the coil, i.e., it will decrease across the slice thickness, and this effect will be more pronounced for smaller coils.

Basic Absolute SNR Predictions

The prediction is based on the paper by Edelstein et. al., Magn Reson. Med. 3, 604-681 (1986). In this paper they report an ‘intrinsic’ SNR of ˜1000, measured in a volume head coil (a ˜30 cm) in a 0.12 T field strength, normalized to a water volume of 1 cm³ and a bandwidth of 1 Hz. Extrapolating from this setup to a coil size of a=2 mm, will lead to a detection bandwidth of 5,000 Hz, as derived from the need to detect a decaying CPMG echo train in which the echo peaks are separated by 200 μs. Now the question is how to factor the magnetic field strength and the gradient strength. Obviously, the SNR will be at least proportional to the field (actually, for very low frequencies the field dependence will be stronger than linear) and inversely proportional to the gradient because with a limited excitation bandwidth, the gradient will determine the excited slice thickness. The important factor is therefore B₂/G₂, and it turns out that, using the magnet geometry described in paragraph 3 above, this ratio is independent of the field strength. Therefore, without loss of generality, we can choose the same field strength of 0.12 T and calculate the resulting gradient and slice thickness. It turns out that this slice thickness will be ˜0.03 mm, so that the excited volume will be 0.2×0.2×0.003=1.2×10 cm³.

Now we can calculate the predicted SNR as follows:

$$SNR = 4000 • \frac{300}{2} • \frac{1}{\sqrt{5000}} •1 .2 {•10}^{- 4} = 1.018$$

The first factor is due to the smaller coil dimension (‘filling factor’), the second factor accounts for the detection bandwidth, and the third factor for the detected volume. It is possible that the improvement due to the ‘filling factor’ is stronger than given by the ratio of coil diameters; this is certainly the case directly at the coil's surface, but when moving away from the coil the situation may actually be worse than assumed in Eq. [15], so that this assumption may be a good approximation to the average behavior. It should also be kept in mind that the prediction is for the water signal at fill strength, however, if we intend the signal from lipid protons (which constitute only a percentage of the full signal) and with the signal attenuated by diffusion, the predicted SNR could be smaller by at least an order of magnitude.

The calculation applies to a single scan, however applying a pulse sequence of 4 echoes train with 60 averages (total of 1 minute at Time To Repeat, TR=1 Sec), we will achieve an SNR of ˜11:1 for the full water signal.

The inclusion of a correlation/prediction technique for enhancement of the signal to noise ratio of the NMR signals sampled by the NMR probe, will drive the typical acquisition time for obtaining the required data by the MD performing the balloon angioplasty procedure down to 1 second typically, for the same signal to noise ratio.

This correlation prediction technique incorporates correlation of the obtained NMR echo trains peak signals with the predicted decaying function due to the NMR diffusion coefficients in fat and other soft tissues related to the imaged zone inside the imaged duct, such as, but not limited to, the coronary artery. The peak of the NMR signals decay in a predictable manner, due to the magnetic field gradient generated inherently by the NMR probe permanent magnet across the scanned object, as given by Equations 6 and 7 above. The more a molecule moves (diffuses) during the NMR scanning cycle, the more signal it will lose as a consequence of that movement, which is taken into account by the data reduction and analysis used to produce these plots.

Table 2 below sets fourth an analysis of the magnetic field strength for three NMR probe magnetization alternatives: Axial, Diametral and Radial described below, as a function of the radial distance from the magnet long axis. The analyzed values along the radial direction were obtained for a magnet cylinder of 5 mm length and 1 mm diameter, both at the axial center and at 1 mm up the axis.

TABLE 2

| | Axial | Axial | Diametral | Diametral | Radial | |
|---|---|---|---|---|---|---|
| | at center | at +1 mm | at center | at +1 mm | at center | Radial at +1 mm |
| R [mm] | B [mT] | B [mT] | B [mT] | B [mT] | B [mT] | B [mT] |
| 0.5 | 1.72E+01 | 2.61E+01 | 7.66E+02 | 6.00E+02 | 2.94E+02 | 3.14E+02 |
| 0.6 | 1.68E+01 | 2.51E+01 | 3.21E+02 | 3.25E+02 | 8.22E−01 | 4.23E+00 |
| 0.7 | 1.63E+01 | 2.41E+01 | 2.38E+02 | 2.41E+02 | 9.15E−01 | 3.93E+00 |
| 0.8 | 1.58E+01 | 2.29E+01 | 1.84E+02 | 1.86E+02 | 9.92E−01 | 3.63E+00 |
| 0.9 | 1.53E+01 | 2.18E+01 | 1.46E+02 | 1.48E+02 | 1.05E+00 | 3.33E+00 |
| 1 | 1.47E+01 | 2.06E+01 | 1.19E+02 | 1.21E+02 | 1.10E+00 | 3.05E+00 |
| 1.1 | 1.41E+01 | 1.94E+01 | 9.94E+01 | 1.00E+02 | 1.13E+00 | 2.78E+00 |
| 1.2 | 1.35E+01 | 1.82E+01 | 8.41E+01 | 8.44E+01 | 1.15E+00 | 2.54E+00 |
| 1.3 | 1.29E+01 | 1.71E+01 | 7.21E+01 | 7.21E+01 | 1.15E+00 | 2.31E+00 |
| 1.4 | 1.23E+01 | 1.60E+01 | 6.24E+01 | 6.22E+01 | 1.14E+00 | 2.10E+00 |
| 1.5 | 1.17E+01 | 1.49E+01 | 5.46E+01 | 5.42E+01 | 1.13E+00 | 1.91E+00 |
| 1.6 | 1.11E+01 | 1.39E+01 | 4.81E+01 | 4.76E+01 | 1.10E+00 | 1.75E+00 |
| 1.7 | 1.05E+01 | 1.30E+01 | 4.27E+01 | 4.21E+01 | 1.07E+00 | 1.59E+00 |
| 1.8 | 9.98E+00 | 1.21E+01 | 3.81E+01 | 3.74E+01 | 1.03E+00 | 1.46E+00 |
| 1.9 | 9.44E+00 | 1.13E+01 | 3.42E+01 | 3.34E+01 | 9.95E−01 | 1.33E+00 |
| 2 | 8.92E+00 | 1.05E+01 | 3.08E+01 | 3.00E+01 | 9.53E−01 | 1.22E+00 |
| 2.1 | 8.42E+00 | 9.77E+00 | 2.79E+01 | 2.71E+01 | 9.09E−01 | 1.12E+00 |
| 2.2 | 7.94E+00 | 9.11E+00 | 2.53E+01 | 2.45E+01 | 8.64E−01 | 1.03E+00 |
| 2.3 | 7.49E+00 | 8.50E+00 | 2.31E+01 | 2.23E+01 | 8.20E−01 | 9.49E−01 |
| 2.4 | 7.06E+00 | 7.93E+00 | 2.11E+01 | 2.03E+01 | 7.75E−01 | 8.75E−01 |
| 2.5 | 6.65E+00 | 7.40E+00 | 1.93E+01 | 1.85E+01 | 7.32E−01 | 8.07E−01 |
| 2.6 | 6.27E+00 | 6.92E+00 | 1.77E+01 | 1.70E+01 | 6.89E−01 | 7.45E−01 |
| 2.7 | 5.91E+00 | 6.47E+00 | 1.63E+01 | 1.56E+01 | 6.49E−01 | 6.89E−01 |
| 2.8 | 5.57E+00 | 6.05E+00 | 1.51E+01 | 1.44E+01 | 6.10E−01 | 6.38E−01 |
| 2.9 | 5.25E+00 | 5.67E+00 | 1.39E+01 | 1.33E+01 | 5.72E−01 | 5.91E−01 |
| 3 | 4.95E+00 | 5.31E+00 | 1.29E+01 | 1.23E+01 | 5.37E−01 | 5.48E−01 |
| 3.1 | 4.67E+00 | 4.98E+00 | 1.20E+01 | 1.14E+01 | 5.03E−01 | 5.08E−01 |
| 3.2 | 4.40E+00 | 4.68E+00 | 1.11E+01 | 1.06E+01 | 4.72E−01 | 4.72E−01 |
| 3.3 | 4.16E+00 | 4.39E+00 | 1.03E+01 | 9.84E+00 | 4.42E−01 | 4.39E−01 |
| 3.4 | 3.92E+00 | 4.13E+00 | 9.64E+00 | 9.17E+00 | 4.14E−01 | 4.08E−01 |
| 3.5 | 3.71E+00 | 3.89E+00 | 8.99E+00 | 8.56E+00 | 3.87E−01 | 3.80E−01 |
| 3.6 | 3.50E+00 | 3.66E+00 | 8.40E+00 | 8.00E+00 | 3.63E−01 | 3.54E−01 |
| 3.7 | 3.31E+00 | 3.45E+00 | 7.86E+00 | 7.48E+00 | 3.40E−01 | 3.30E−01 |
| 3.8 | 3.13E+00 | 3.26E+00 | 7.37E+00 | 7.02E+00 | 3.18E−01 | 3.08E−01 |
| 3.9 | 2.97E+00 | 3.07E+00 | 6.91E+00 | 6.58E+00 | 2.98E−01 | 2.88E−01 |
| 4 | 2.81E+00 | 2.90E+00 | 6.49E+00 | 6.19E+00 | 2.79E−01 | 2.69E−01 |
| 4.1 | 2.66E+00 | 2.75E+00 | 6.10E+00 | 5.82E+00 | 2.62E−01 | 2.52E−01 |
| 4.2 | 2.53E+00 | 2.60E+00 | 5.75E+00 | 5.48E+00 | 2.46E−01 | 2.36E−01 |
| 4.3 | 2.40E+00 | 2.46E+00 | 5.41E+00 | 5.17E+00 | 2.31E−01 | 2.21E−01 |
| 4.4 | 2.28E+00 | 2.33E+00 | 5.11E+00 | 4.88E+00 | 2.16E−01 | 2.07E−01 |
| 4.5 | 2.16E+00 | 2.21E+00 | 4.82E+00 | 4.61E+00 | 2.03E−01 | 1.94E−01 |

### DESCRIPTION OF SEVERAL PREFERRED EMBODIMENTS

The NMR probe illustrated in FIGS. 1 and 2, referred to in the above description of the basic principles of operation of the present invention, represents one preferred embodiment of the invention. In this embodiment, the permanent magnet 2 is of a solid cylindrical construction generating a diametrical or transverse magnetic field B₀ with respect to the probe longitudinal axis 5. As described, the two coils 3, 4 are orthogonally located with respect to each other such that the gradient magnetic field B₁ is rotatable about the probe axis 5 according to the relative currents in the two coils. The two orthogonal coils thus steer the gradient magnetic field B₁ in azimuth to select the sector being imaged, while the radial distance from the magnet is selected by the frequency of the current supplied to the two coils. As indicated earlier, and as to be described more particularly below, such an NMR probe is particularly useful for intra-luminal imaging, e.g., for measuring the azimuthal distribution of the plaque in a narrowed blood vessel, and/or for determining whether the plaque contains a dangerously large amount of lipids.

The foregoing stricture and operation of an NMR probe in accordance with the present invention are to be sharply distinguished from the NMR probe described in the above-cited U.S. Pat. No. 5,390,673. The probe described in that patent includes adjustable steering fields positioned around the front of the magnet for Steering the main field of the magnetic in the longitudinal direction, rather than in the axial or transverse direction. Accordingly, such a probe would not be suitable for intra-luminal imaging.

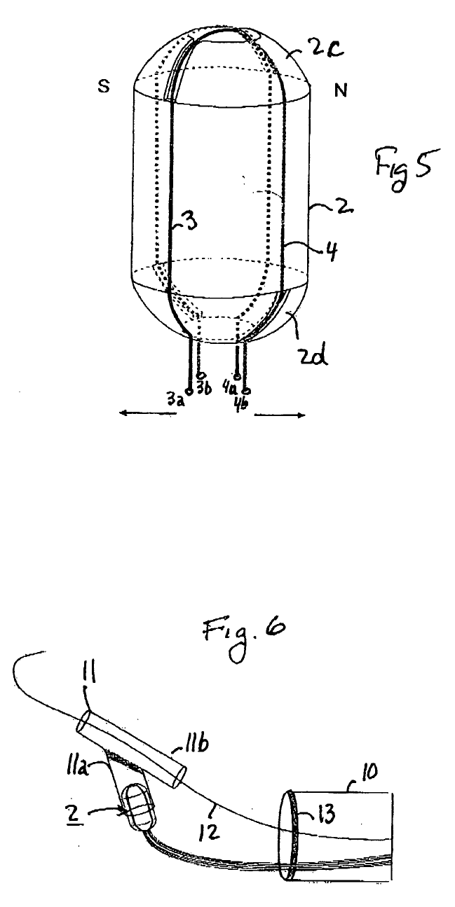

FIG. 4 illustrates an example of an electrical Circuit that may be used for driving the two coils 3, 4 in the probe of FIG. 1. Thus, both coils may be driven by a common RF signal source 6, with the circuit from signal source 6 to coil 3 provided with a variable resistor 7 and with a switch-over switch 8 in order to control the relative currents applied to the two coils.

FIG. 5 illustrates a preferred construction of the NMR probe of FIGS. 1 and 2 for use in intra-luminal imaging; whereas FIG. 6 illustrates the manner in which such a probe may be used for that purpose. Thus, as shown in FIG. 5, the permanent magnet 2 is of a dome-shaped configuration at its opposite ends 2c, 2d to facilitate the introduction of the probe into the lumen. As shown in FIG. 6, the probe 2 is introduced into the lumen via a catheter 10 by means of a bifurcated sleeve 11. This is dune by inserting the probe 2 into one bifurcation 11a of sleeve 11 while using the other bifurcation 11b for guiding the sleeve, and the probe therein, through the catheter by means of a guidewire 12.

For example, the bifurcated sleeve 11 may be a very thin and flexible sleeve of Teflon™, and the guidewire 12 may be a wire of 0.3 mm. Catheter 10 may include a marking ring 13, e.g., of 3 mm.

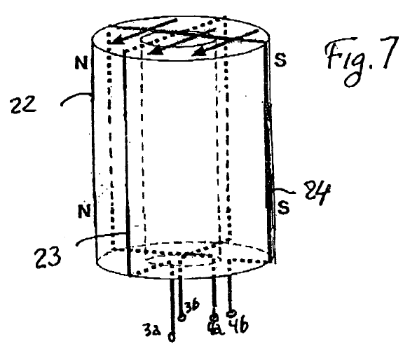

FIGS. 7-11 illustrate variations in the constructions of the permanent magnet and in the direction of the homogenous magnetic field (B₀) generated by it.

Thus, in the NMR probe illustrated in FIG. 7, the permanent magnet, therein designated 22, is of a hollow cylindrical configuration, rather than of a solid cylindrical configuration as in FIGS. 1 and 2, but the homogenous magnetic field B₀ generated by it is also a diametrical field as in FIGS. 1 and 2. It is to be noted that in this embodiment, the two RF coils 23, 24 are also orthogonally located with respect to each other and have axes concentric with the longitudinal axis of the magnet and of the probe.

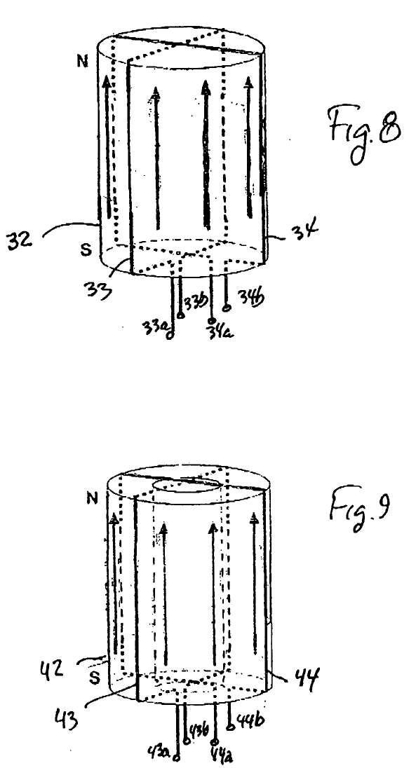

In the probe of FIG. 8, the permanent magnetic 32 is of a solid cylindrical configuration, but it generates an axial homogenous magnetic field B₀, rather than a diametrical magnetic field as in FIGS. 2 and 7. The orthogonal RF coils are shown at 33 and 34.

In the probe of FIG. 9, the permanent magnet 42 is of a hollow cylindrical configuration, as in FIG. 7, but generates an axial homogenous magnetic field B₀ as in FIG. 8. The orthogonal RF coils are shown at 43 and 44.

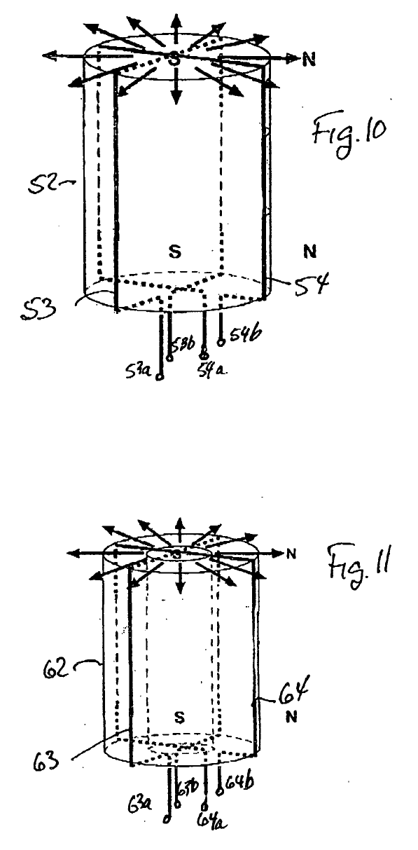

In the probe illustrated in FIG. 10, the permanent magnet 52 is of a solid cylindrical configuration and generates a homogenous magnetic field B₀ in the radial direction; whereas in the probe illustrated in FIG. 11, the permanent magnetic 62 is of a hollow cylindrical configuration and also generates a homogenous magnetic field B₀ in the radial direction. The orthogonal RF coils are shown at 53, 54 and 63, 64, respectively.

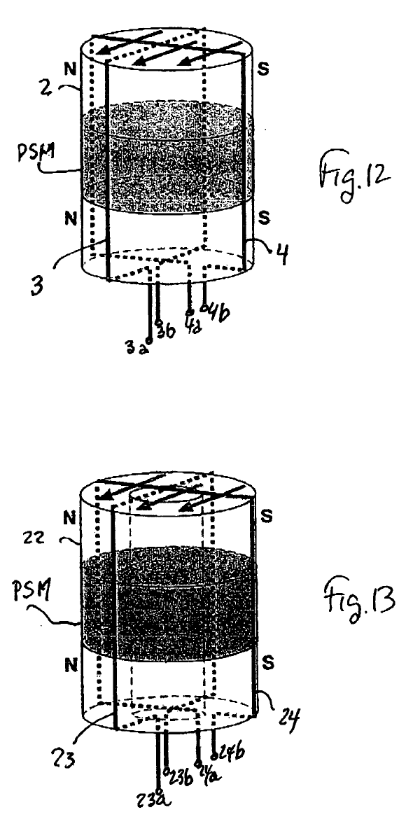

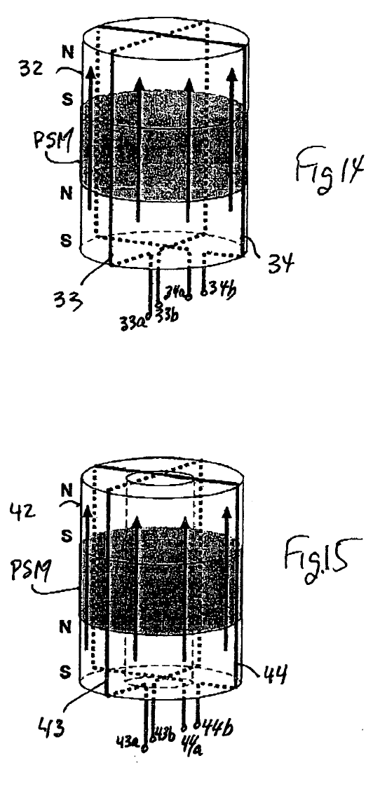

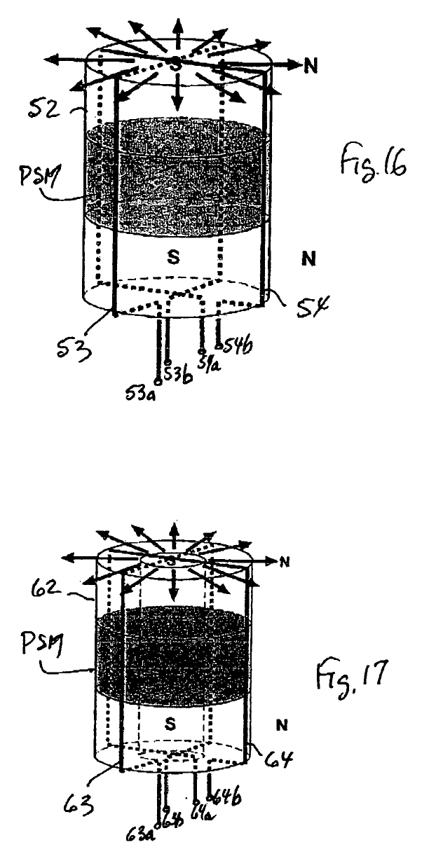

FIGS. 12-17 illustrate NMR probes of similar constructions as described above with respect to FIGS. 2 and 7-11, respectively, except that the probes include partially saturated magnet (PSM) segments. It is believed that such a modification in the construction of the probe enables the probe to be radically improved by increasing its sensitivity (hence, the achievable SNR and spatial resolutions by over two orders of magnitude.

A partially saturated permanent magnet will have a permeability which is between the permeability of the non-magnetized material (typically μᵣ˜5000, for Nd—Fe—B magnets), and the permeability of the fully magnetized magnet (typically μᵣ=1.05). The static magnetic Stray field (like the homogenous magnetic Bₒ) around such a magnet will be proportional to the degree of saturation, through the so-called remnant magnetization Jᵣ. Therefore, if one only considers the implications for Bₒ, the use of PSM may appear to be of disadvantage for the probe performance, since the NMR detection sensitivity is known to improve with increasing magnetic field Bₒ. However, when one considers the entire design of the NMR probe assembly, as illustrated in FIGS. 12-17, which includes the magnet segments and the RF coils, the advantage for incorporating PSM becomes apparent.

This advantage is rooted in the perpendicular (to Bₒ) magnetic field B₁, created by passing current through the RF coils. The value of this field can be calculated from the so-called Biot-Savart law, which relates the magnetic field induced by an electric current flowing in a wire:

$$d {B}^{->} ( p ) = \frac{μ}{4 π} \frac{Id \overline{L} x {r}^{->}}{{\vert {r}^{->} \vert}^{3}} \qquad [ 1 ]$$

where d\vec{B}(p) is the differential contribution to the magnetic field at some location p, induced by an electric current I, flowing in a small segment of wire whose length and orientation are defined by the vector \overline{L}, and \overline{r} is the vector connecting between the wire segment and point p. The magnetic field is proportional to the permeability μ. For the case that the coil is wound around non-magnetic material, the permeability is that of free space (μₒ), but for the case of magnetic material the permeability is increased (relative to μₒ) by the factor μᵣ. Our calculations indicate that the value of μᵣ, for a Nd—Fe—B magnet saturated to about 50% (i.e., with Jᵣ˜0.5 T, instead of the maximal attainable value of 1 T) will be about 300. Therefore, a roughly 300-fold increase can be expected in the value of B₁ generated by a coil wound around a PSM over the value generated by the same current by a coil wound around a non-magnetic material, or a fully saturated magnet.

This has very dramatic consequences for the performance of the magnet-coil assembly. Not only will the power needed to produce the NMR excitation pulses be drastically reduced (or their time shortened), but the sensitivity of the RF coil as receiver will also be improved, by virtue of the reciprocity principle, which states that this sensitivity is proportional to the ration B_{I}/I.

FIGS. 12-17 illustrate NMR probes constructed as described above with respect to FIGS. 2 and 7-11, respectively, but including the above-described partially saturated magnet (PSM) segments. For the sake of brevity and to facilitate understanding, the same reference numerals have been used in FIGS. 12-17 as in FIGS. 2 and 7-11, respectively, but the partially saturated magnet segments have been designated PSM.

While the invention has been described with respect to several preferred embodiments, it will be appreciated that these are set forth merely for purposes of example, and that many other variations, modifications and applications of the invention may be made.

What is claimed is:

1. An NMR probe, comprising:
a permanent magnet generating a homogenous magnetic field (B₀);
and a pair of RF gradient coils enclosing said permanent magnet for receiving RF current to generate a gradient magnetic field (B₁) perpendicular to said homogenous magnetic field (B₀);
said pair of RF coils being orthogonally located with respect to each other such that the gradient magnetic field (B₁) is rotatable about the longitudinal axis of the probe according to the current through one RF coil relative to that through the other RF coil.

2. The probe according to claim 1, wherein said RF coils are located along the outer surface of said permanent magnet orthogonal to each other and have longitudinal axes parallel to the longitudinal axis of said probe.

3. The probe according to claim 1, wherein said permanent magnet and said pair of RF coils are sized and configured for introduction into a lumen of a person's body.

4. The probe according to claim 3, wherein said permanent magnet is of a dome-shaped configuration at its opposite ends to facilitate the introduction of the probe into said lumen.

5. The probe according to claim 1, wherein said probe further comprises an RF current supply having means for varying the RF current supplied to each of said RF coils.

6. The probe according to claim 1, wherein said permanent magnet is of a solid cylindrical configuration and generates a diametrical magnetic field B₀.

7. The probe according to claim 1, wherein said permanent magnet is of a hollow cylindrical configuration and generates a diametrical magnetic field B₀.

8. The probe according to claim 1, wherein said permanent magnet is of a solid cylindrical configuration and generates an axial magnetic field B₀.

9. The probe according to claim 1, wherein said permanent magnet is of a hollow cylindrical configuration and generates an axial magnetic field B₀.

10. The probe according to claim 1, wherein said permanent magnet is of a solid cylindrical configuration and generates a radial magnetic field B₀.

11. The probe according to claim 1, wherein said permanent magnet is of a hollow cylindrical configuration and generates a radial magnetic field B₀.

12. The probe according to claim 1, wherein said permanent magnet is of a permanent magnetic material for the complete length of the probe.

13. The probe according to claim 1, wherein said permanent magnet includes an end section of a permanent magnetic material at each of the opposite ends of the probe, and an intermediate section of an unsaturated, high-permeability material between said end sections.

14. An NMR probe, comprising:
a permanent magnet generating a homogenous magnetic field (B₀);
and a pair of RF gradient coils located on the outer surface of said permanent magnet for receiving RF current to generate a gradient magnetic field (B₁) perpendicular to said static magnetic gradient field (B₀);
said pair of RF coils being orthogonally located with respect to each other and have longitudinal axes parallel to the longitudinal axis of said probe, such that the gradient magnetic field (B₁) is rotatable about the longitudinal axis of the probe according to the current through one RF coil relative to that through the other RF coil.

15. The probe according to claim 14, wherein said permanent magnet and said pair of RF coils are sized and configured for introduction into a lumen of a person's body.

16. The probe according to claim 14, wherein said permanent magnet is of a dome-shaped configuration at its opposite ends to facilitate the introduction of the probe into said lumen.

17. The probe according to claim 14, wherein said permanent magnet is of a permanent magnetic material for the, complete length of the probe.

18. The probe according to claim 14, wherein said permanent magnet includes an end section of a permanent magnetic material at each of the opposite ends of the probe, and an intermediate section of an unsaturated, high-permeability material between said end sections.

19. An NMR probe, comprising:
a permanent magnet generating a homogenous magnetic field (B₀);
and a pair of RF gradient coils located on the outer surface of said permanent magnet for receiving RF current to generate a gradient magnetic field (B₁) perpendicular to said static magnetic gradient field (B₀);
said pair of RF coils being orthogonally located with respect to each other and have longitudinal axes parallel to the longitudinal axis of said probe, such that the gradient magnetic field (B₁) is rotatable about the longitudinal axis of the probe according to the current through one RF coil relative to that through the other RF coil;
said permanent magnet and said pair of RF coils being sized and configured for introduction into a lumen of a person's body.

20. A catheter for introduction into a lumen of a person's body comprising: a bifurcated sleeve having one bifurcation receiving the probe defined in claim 19, and another bifurcation for receiving a guidewire for guiding the probe to a desired location within said lumen.
